(19) United States
(12) Patent Application Publication
(10) Pub. No.: US 2025/0082421 A1
(43) Pub. Date: Mar. 13, 2025

(54) SYSTEMS AND METHODS FOR ADJUSTING AN INSTRUMENT HOLDER WHILE MAINTAINING A POSITION OF A REMOTE CENTER OF MOTION

(71) Applicant: INTUITIVE SURGICAL OPERATIONS, INC., Sunnyvale, CA (US)

(72) Inventor: Carolyn M. Fenech, Campbell, CA (US)

(21) Appl. No.: 18/827,407

(22) Filed: Sep. 6, 2024

Related U.S. Application Data
(60) Provisional application No. 63/581,161, filed on Sep. 7, 2023.

(51) Int. Cl. A61B 34/30; A61B 34/20; A61B 90/00

(52) U.S. Cl. CPC A61B 34/30; A61B 2034/2059; A61B 2034/302; A61B 2090/062; A61B 2090/064; A61B 2090/0807

(57) ABSTRACT

A method for operating a medical system comprises inserting a cannula through a body wall of a patient to a target depth, adjusting an adjustable connecting mechanism to a target setting to position an instrument holder at a target position relative to a robotic manipulator arm, and coupling the instrument holder to the cannula. The adjustable connecting mechanism comprises a plurality of depth position settings to adjust a position of the instrument holder relative to the robotic manipulator arm based on the target depth. The target setting corresponds to the target depth, and a remote center distance between the robotic manipulator arm and a remote center of motion is maintained as the adjustable connecting mechanism moves the instrument holder to the target position relative to the robotic manipulator arm. The cannula pivots about the remote center of motion.

### CROSS-REFERENCED APPLICATIONS

This application claims priority to and the benefit of U.S. Provisional Application No. 63/581,161, filed Sep. 7, 2023, and entitled “Systems and Methods for Adjusting an Instrument Holder While Maintaining a Position of a Remote Center of Motion,” which is incorporated by reference herein in its entirety.

### FIELD

Examples described herein relate to systems and methods for adjusting an instrument holder of a hardware centered manipulator system while maintaining a remote center position.

### BACKGROUND

In hardware centered manipulator systems, the remote center of motion (the point about which an instrument holder, such as a cannula, pivots) is set based on the configuration of the manipulator arm. In existing systems, a cannula may pivot about the remote center of motion but may have a fixed insertion depth such that a length of the cannula above and below the remote center of motion is unchanged during a procedure or between different procedures. Sometimes, the insertion depth of the cannula relative to the remote center of motion may lead to collisions between the manipulator arm and the body wall or collisions between manipulator arms. With existing systems, a clinician may be unable to perform an operation close to the distal end of the cannula (and closer to the inner body wall surface) without interfering with the body wall and thus may attempt to adjust the cannula insertion depth during a medical procedure. Improved systems and methods are needed to adjust an anatomical interaction location of the cannula (the location along the length of the cannula where the cannula meets the remote center) to reduce the likelihood of injury to the body wall during a medical procedure and/or based on the surgeon's preferences, for example.

### SUMMARY

Various features may allow for a position of an instrument holder frame to be adjusted based on an insertion depth of a cannula into a body wall of a patient. The adjustable position of the instrument holder frame allows for the position of a remote center to be maintained relative to the patient body wall while allowing for the cannula to be positioned at different insertion depths according to user preference and/or clinical needs. The following presents a simplified summary of various examples described herein and is not intended to identify key or critical elements or to delineate the scope of the claims.

Consistent with some examples, a method for operating a medical system includes inserting a cannula through a body wall of a patient to a target depth, adjusting an adjustable connecting mechanism to a target setting to position an instrument holder at a target position relative to a robotic manipulator arm, and coupling the instrument holder to the cannula. The adjustable connecting mechanism includes a plurality of depth position settings to adjust a position of the instrument holder relative to the robotic manipulator arm based on the target depth. The target setting corresponds to the target depth, and a remote center distance between the robotic manipulator arm and a remote center of motion is maintained as the adjustable connecting mechanism moves the instrument holder to the target position relative to the robotic manipulator arm. The cannula pivots about the remote center of motion.

Consistent with other examples, a system includes a robotic manipulator arm and an instrument holder coupled to the robotic manipulator arm by an adjustable connecting mechanism. The system further includes a processing system including one or more processors and a memory having computer readable instructions stored thereon which, when executed by the one or more processors, cause the processing system to provide user instructions for inserting a cannula through a body wall of a patient to a target depth. The instructions further cause the processing system to provide user instructions for adjusting the adjustable connecting mechanism to a first setting to position the instrument holder at a first position relative to the robotic manipulator arm. The adjustable connecting mechanism includes a plurality of depth position settings to adjust a position of the instrument holder relative to the robotic manipulator arm based on the target depth. The first setting corresponds to the target depth, and a remote center distance between the robotic manipulator arm and a remote center of motion is maintained as the adjustable connecting mechanism moves the instrument holder to the first position relative to the robotic manipulator arm. The cannula pivots about the remote center of motion. The instructions further cause the processing system to provide user instructions for coupling the instrument holder to the cannula.

Consistent with other examples a system includes a robotic manipulator arm, which includes a hardware constrained remote center of motion, an instrument holder for coupling to an instrument, and a connecting mechanism coupling the robotic manipulator arm to the instrument holder. The system further includes a cannula. The connecting mechanism is configured to adjust a position of the instrument holder relative to the robotic manipulator arm between a first position and a second position. The first position corresponds to a first insertion depth of the cannula in a patient body wall, and the second position corresponds to a second insertion depth of the cannula in the patient body wall. The remote center of motion relative to the patient body wall is maintained when the instrument holder is in the first position and in the second position.

Other examples include corresponding computer systems, apparatus, and computer programs recorded on one or more computer storage devices, each configured to perform the actions of any one or more methods described below.

It is to be understood that both the foregoing general description and the following detailed description are illustrative and explanatory in nature and are intended to provide an understanding of the various examples described herein without limiting the scope of the various examples described herein. In that regard, additional aspects, features, and advantages of the various examples described herein will be apparent to one skilled in the art from the following detailed description.

### BRIEF DESCRIPTIONS OF THE DRAWINGS

FIG. 1 illustrates a manipulator arm coupled to an instrument holder and a cannula according to some examples.

FIG. 2A illustrates an instrument holder positioned at a target position relative to a manipulator arm according to some examples.

FIG. 2B illustrates an instrument holder positioned at a target position relative to a manipulator arm according to some examples.

FIG. 3A illustrates an adjustable connecting mechanism including a lead screw according to some examples.

FIG. 3B illustrates an adjustable connecting mechanism including a rack and pinion according to some examples.

FIG. 3C illustrates an adjustable connecting mechanism including a ratchet according to some examples.

FIG. 4 is a flowchart illustrating a method of positioning an instrument holder at a target position according to some examples.

FIG. 5 is a simplified diagram of a computer-assisted, teleoperated system according to some examples.

Various examples described herein and their advantages are described in the detailed description that follows. It should be appreciated that like reference numerals are used to identify like elements illustrated in one or more of the figures for purposes of illustrating but not limiting the various examples described herein.

### DETAILED DESCRIPTION

In the following description, specific details describe some examples consistent with the present disclosure. Numerous specific details are set forth in order to provide a thorough understanding of the examples. It will be apparent to one skilled in the art, however, that some examples may be practiced without some or all of these specific details. The specific examples disclosed herein are meant to be illustrative but not limiting. One skilled in the art may realize other elements that, although not specifically described, are within the scope and the spirit of this disclosure. In addition, to avoid unnecessary repetition, one or more features shown and described in association with one example may be incorporated into other examples unless specifically described otherwise or if the one or more features would make an example non-functional. In some instances, well known methods, procedures, components, and circuits have not been described in detail so as not to unnecessarily obscure aspects of the examples.

In some examples, an adjustable connecting mechanism may couple an instrument holder to a manipulator arm (e.g., a robotic manipulator arm) of a manipulator assembly. The adjustable connecting mechanism may be used to adjust the position of a frame or link of the instrument holder with respect to the manipulator arm. The position of the instrument holder frame or link may be adjusted based on the insertion depth of a cannula into a body wall of a patient. The adjustable position of the instrument holder frame or link allows for the position of a remote center to be maintained relative to the patient body wall while allowing for the cannula to be positioned at different insertion depths according to user preference and/or clinical needs. The remote center may remain fixed relative to the body wall and may be set at the same location regardless of which cannula insertion depth is selected. An adjustable instrument holder frame or link may help to: avoid collisions between the manipulator arm and the body wall; avoid collisions between manipulator arms; allow a physician to perform an operation closer to the distal end of the cannula (and closer to the inner body wall surface) without harming the body wall; and/or reduce the need to manually adjust the cannula insertion depth before and/or during a medical procedure.

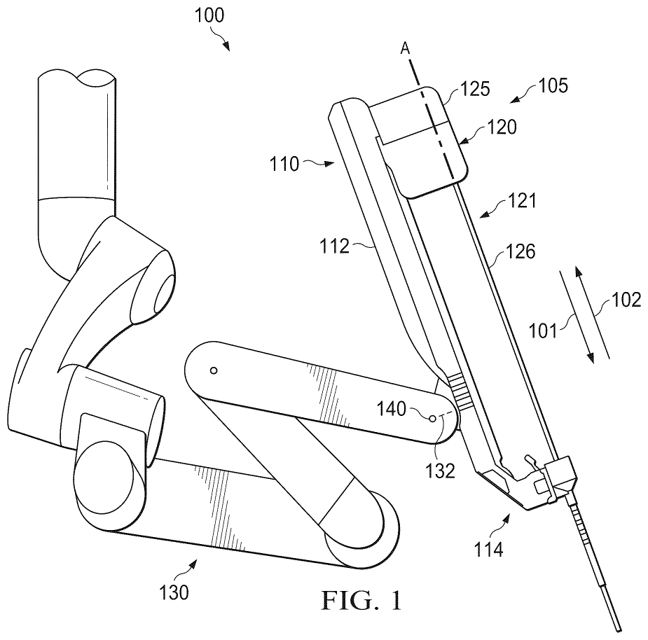

FIG. 1 illustrates an instrument manipulation system 100 including an instrument drive system 105 and a manipulator 130. The instrument drive system 105 includes an instrument holder 110 and a carriage 120. In some examples, the instrument drive system 105 includes an instrument 121 (which may be an elongate instrument) having an instrument housing 125 coupled to an instrument shaft 126. The instrument holder 110 includes an instrument holder frame 112 and a connection portion 114. The carriage 120 may be coupled to the instrument holder frame 112. The carriage 120 may move in a distal direction 101 and a proximal direction 102 relative to the instrument holder frame 112. This motion may also move the instrument 121 (including the instrument housing 125 and the instrument shaft 126) in the distal and proximal directions 101, 102 relative to the instrument holder frame 112. Additionally, the instrument holder 110 may move in the distal and proximal directions 101, 102 relative to the manipulator arm 130. In some examples, when the instrument holder 110 moves in the distal and proximal directions 101, 102, the position of the manipulator arm 130 does not change. The distal and proximal directions 101, 102 may be generally parallel with a longitudinal axis A of the instrument shaft 126.

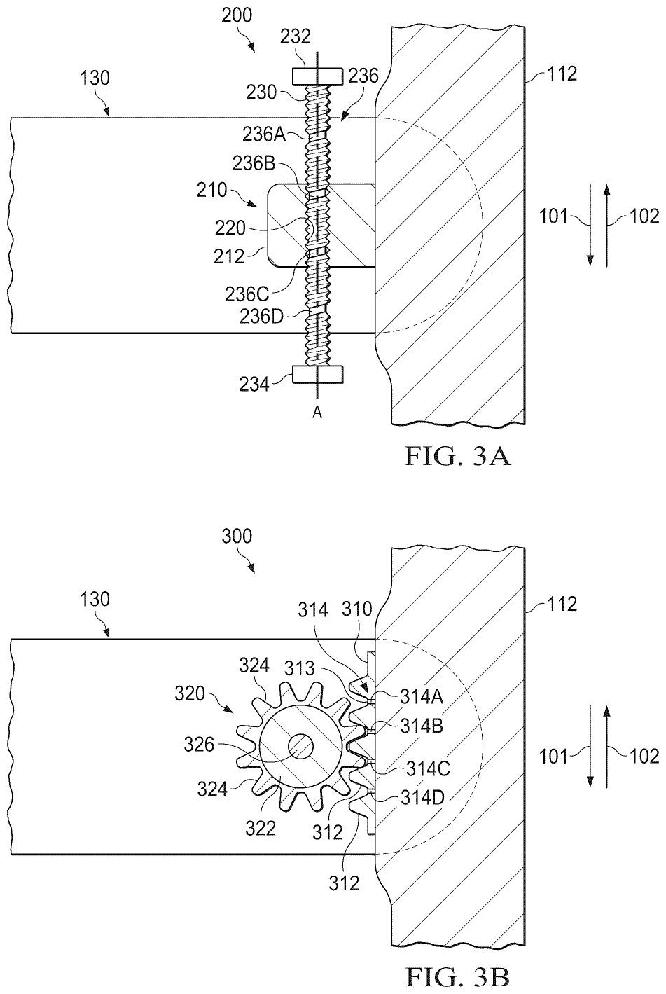

The manipulator 130 (which may be a robotic manipulator arm and/or a manipulator assembly) includes a distal portion 135 and a connection location 140. The connection location 140 is the location where the manipulator arm 130 is coupled to the instrument holder 110. An adjustable connecting mechanism (shown, for example, in FIGS. 3A-3C) may be positioned on or within one or both of the manipulator arm 130 or the instrument holder 110 at the connection location 140. In some examples, the adjustable connecting mechanism couples the manipulator arm 130 to the instrument holder frame 112. The adjustable connecting mechanism may be coupled to the instrument holder frame 112. As will be described in greater detail below, the adjustable connecting mechanism allows for the instrument holder frame 112 to be moved in the distal and proximal directions 101, 102 relative to the manipulator arm 130 while maintaining a distance between the manipulator arm 130 and a remote center of motion.

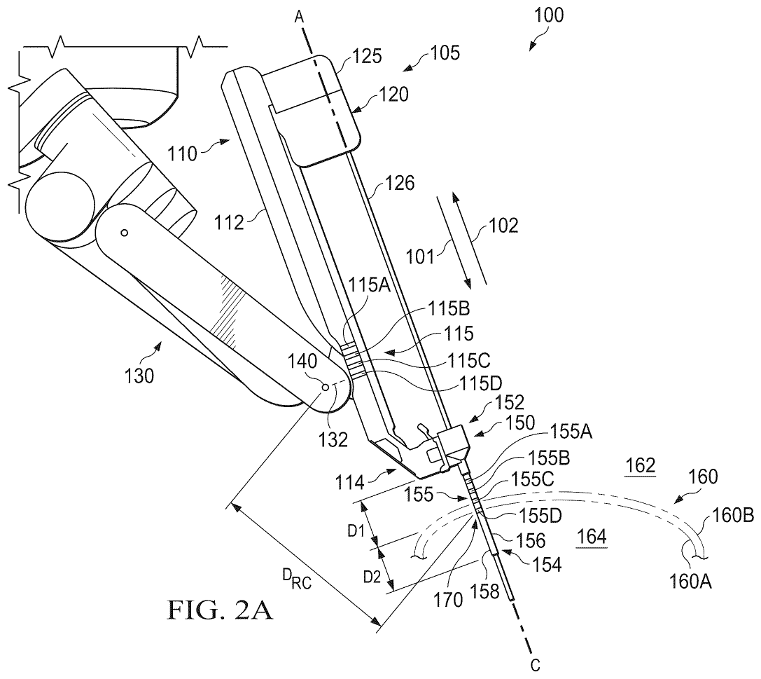

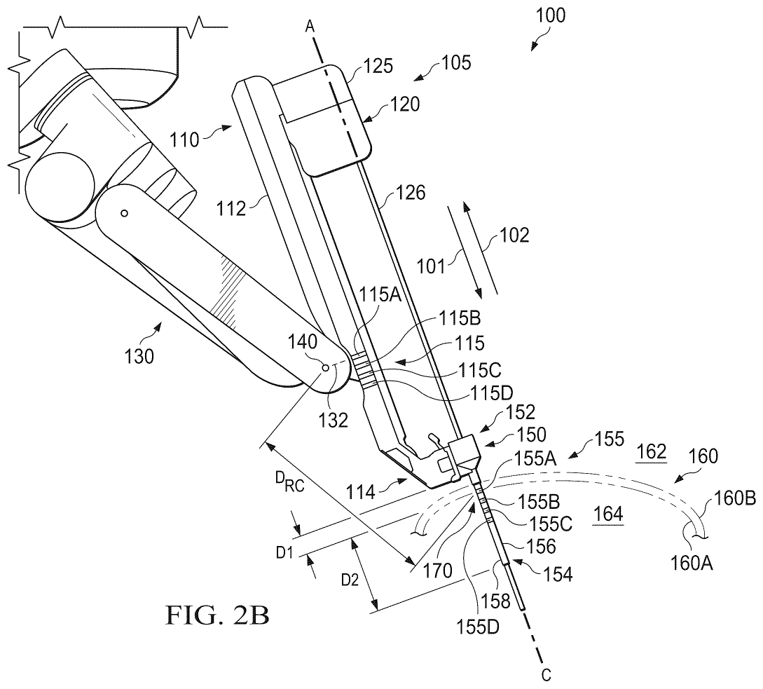

FIG. 2A illustrates the instrument holder 110 positioned at a target position relative to the manipulator arm 130 according to some examples. FIG. 2B illustrates the instrument holder 110 positioned at another target position relative to the manipulator arm 130 according to some examples. As shown in FIGS. 2A and 2B, the connection portion 114 may be coupled to a cannula 150. The cannula 150 includes a proximal portion 152 and a shaft portion 156. The shaft portion includes a distal portion 154, which includes a distal end 158. The cannula 150 may be positioned through a body wall 160 of a patient. When the cannula 150 is positioned through the body wall 160, the proximal portion 152 of the cannula 150 is positioned in an exterior space 162 exterior to the body wall 160 and outside of the patient, and the distal portion 154 of the cannula 150 is positioned within an interior space 164 interior to the body wall 160 and inside of the patient. The body wall 160 includes an inner surface 160A, an outer surface 160B, and a thickness extending between the inner and outer surfaces 160A, 160B. The inner surface 160A borders the interior space 164, and the outer surface 160B borders the exterior space 162.

As shown in FIG. 2A, the instrument shaft 126 may extend through the cannula 150 and into the interior space 164 within the patient. The longitudinal axis A of the instrument shaft 126 may be generally parallel with a longitudinal axis C of the cannula 150. The instrument holder frame 112 includes a plurality of markings or indicators 115. The plurality of indicators 115 includes four individual indicators 115A, 115B, 115C, and 115D. Any other number of indicators may be included in the plurality of indicators 115. As shown in FIG. 2A, the indicator 115A is the most proximal indicator of the plurality of indicators 115, and the indicator 115D is the most distal indicator of the plurality of indicators 115. The indicator 115B is distal to the indicator 115A and proximal to the indicator 115C. The indicator 115C is proximal to the indicator 115D. In some examples, the manipulator arm 130 includes a base marking 132. As shown in FIG. 2A, the base marking 132 may be aligned with or otherwise fixed relative to the connection location 140 of the manipulator arm 130. For example, FIG. 2A shows the base marking 132 aligned with the indicator 115D. In some examples, the base marking 132 may be aligned with the instrument holder frame 112 at any other location, such as locations proximal to the indicator 115A, distal to the indicator 115D, or between the indicators 115A, 115B, 115C, and 115D.

The shaft portion 156 of the cannula 150 may include a plurality of markings 155. The plurality of markings 155 may include four individual markings 155A, 155B, 155C, and 155D. Any other number of markings may be included in the plurality of markings 155. As shown in FIG. 2A, the marking 155A is the most proximal marking of the plurality of markings 155, and the marking 155D is the most distal marking of the plurality of markings 155. The marking 155B is distal to the marking 155A and proximal to the marking 155C. The marking 155C is proximal to the marking 155D.

Each of the indicators 115 on the instrument holder frame 112 may correspond to one of the markings 155 on the cannula 150. For example, the most proximal indicator 115A of the indicators 115 corresponds to the most proximal marking 155A of the markings 155. The most distal indicator 115D of the indicators 115 corresponds to the most distal marking 155D of the markings 155. Similarly, the indicator 115B corresponds to the marking 155B, and the indicator 115C corresponds to the marking 155C. In some examples, the indicators 115 and the markings 155 may be labeled with different graphical labels including colors, symbols, textual, and/or numerical labels. Corresponding indicators and markings may be the same type of graphical label. For example, as shown in FIGS. 2A and 2B, the indicator 115A and the marking 155A may each be red; the indicator 115B and the marking 155B may each be blue; the indicator 115C and the marking 155C may each be green; and the indicator 115D and the marking 155D may be each be yellow. These colors are exemplary only, and any other colors may be used for any of the corresponding pairs of indicators and markings. Additionally or alternatively, the indicators 115 and the markings 155 may be labeled with different graphical labels, such as different numbers (e.g., 1, 2, 3, and 4), patterns (e.g., dotted lines, dashed lines, etc.), shapes, textual labels, or any combination thereof. Any other type of graphical labels may be used to identify the indicators 115 and the markings 155.

In some examples, the user may determine a preferred cannula insertion depth of the cannula 150 into the body wall 160 of a patient, which may be a target depth or a target insertion depth. The cannula insertion depth is illustrated in FIG. 2A by the distance D₂. The cannula insertion depth D₂ is measured from a remote center of motion 170 to the distal end 158 of the cannula 150 in a direction parallel with the longitudinal axis C of the cannula 150. For example, the user may determine which marking of the individual markings 155A-D on the cannula 150 will be positioned at the remote center 170. When the desired marking of the markings 155A-D is positioned at the remote center 170, the cannula insertion depth D₂ represents the target insertion depth.

The remote center 170 is the point about which the cannula 150 pivots (e.g., the pivot point of the cannula 150) relative to the body. The remote center 170 position is fixed with respect to the body wall 160 of the patient (e.g., in X, Y, Z Cartesian space) and is set in the same location relative to the body wall 160 regardless of which cannula insertion depth D₂ is chosen. The remote center 170 remains pivotable relative to the fixed position. The location on the cannula 150 that is positioned at the remote center 170 may be an anatomical interaction location of the cannula 150. In some examples, the remote center 170 is a hardware constrained remote center of motion. The location of the remote center 170 may be set based on the configuration of any one or more of the manipulator arm 130, the instrument drive system 105, and/or any other components of the instrument manipulation system 100.

Additionally or alternatively, the remote center 170 may be a software constrained remote center of motion. In some examples, the location of the remote center 170 may be set based on data received by a control system of the manipulation system 100, such as position data, force data, or any other data related to the patient, the procedure to be performed, and the components of the manipulation system 100.

In some examples, the remote center 170 position is in the middle of the body wall 160. In some examples, as shown in FIGS. 2A and 2B, the remote center 170 position is within the body wall 160 and is closer to the inner surface 160A of the body wall 160 than the outer surface 160B of the body wall 160. Alternatively, the remote center 170 position may be closer to the outer surface 160B than the inner surface 160A. In some examples, the remote center 170 position is exterior to the body wall 160 and is located in the exterior space 162 proximal to the outer surface 160B of the body wall 160.

In some examples, the cannula insertion depth D₂ may be adjusted for clinical reasons, to accommodate operating room constraints, and/or based on user preferences. A shallower cannula insertion depth may allow for procedures to be performed closer to the body wall 160 of the patient. In some examples, a deeper cannula insertion depth may allow the instrument shaft 126 to reach deeper into the patient body. The cannula insertion depth D₂ may be adjusted by adjusting the anatomical interaction location of the cannula 150.

When the target cannula insertion depth D₂ is chosen, the instrument holder 110 may be adjusted relative to the manipulator arm 130. Being able to adjust the instrument holder 110 relative to the manipulator arm 130 allows for the position of the remote center 170 to be maintained relative to the body wall 160 when the cannula 150 is positioned at different insertion depths. The instrument holder 110 may be adjusted so that the indicator on the instrument holder frame 112 that corresponds to the marking on the cannula 150 positioned at the remote center 170 is aligned with the base marking 132 on the manipulator arm 130. When the corresponding indicator on the instrument holder frame 112 is aligned with the base marking 132, the instrument holder 110 is positioned at a target position relative to the manipulator arm 130.

For example, as shown in FIG. 2A, the marking 155D on the cannula 150 is positioned at the remote center 170. As discussed above, the indicator 115D corresponds to the marking 155D. The instrument holder 110 may be adjusted so that the indicator 115D is aligned with the base marking 132, as shown in FIG. 2A. In the example of FIG. 2A, when the indicator 115D is aligned with the base marking 132, the instrument holder 110 is at a target position relative to the manipulator arm 130.

FIG. 2B illustrates the cannula 150 positioned at a different cannula insertion depth than the cannula insertion depth shown in FIG. 2A. FIG. 2B illustrates the cannula 150 positioned deeper into the patient than in FIG. 2A. For example, the cannula insertion depth D₂ in FIG. 2B is greater than the cannula insertion depth D₂ in FIG. 2A. As further shown in FIG. 2B, the marking 155A on the cannula 150 is positioned at the remote center 170. As discussed above, the indicator 115A corresponds to the marking 155A. The instrument holder 110 may be adjusted so that the indicator 115A is aligned with the base marking 132 on the manipulator arm 130, as shown in FIG. 2B. In the example of FIG. 2B, when the indicator 115A is aligned with the base marking 132, the instrument holder 110 is at a second target position relative to the manipulator arm 130. The second target position is different than the target position shown in FIG. 2A.

As discussed above, in FIG. 2B, the instrument holder 110 has been moved in the distal direction 101 from the position shown in FIG. 2A to the position shown in FIG. 2B. This may further be shown by comparing a distance D₁ between the connection portion 114 of the instrument holder 110 and the remote center 170. D₁ in FIG. 2A is greater than D₁ in FIG. 2B because the instrument holder 110 is farther away from the remote center 170 in FIG. 2A than in FIG. 2B. When the instrument holder 110 is moved in the distal direction 101, the instrument drive system 105, including the carriage 120 and the instrument 121, may also move in the distal direction 101.

During any adjustment of the instrument holder 110, the position of the manipulator arm 130 remains the same, as shown in FIGS. 2A and 2B. The adjustments to the position of the instrument holder 110 are made relative to the manipulator arm 130. Therefore, a remote center distance D_{RC}, which is a distance between the connection location 140 of the manipulator arm 130 and the remote center 170, remains constant regardless of where the instrument holder 110 is positioned. As shown in FIGS. 2A and 2B, the remote center distance D_{RC} is the same when the instrument holder 110 is positioned at two different positions relative to the manipulator arm 130.

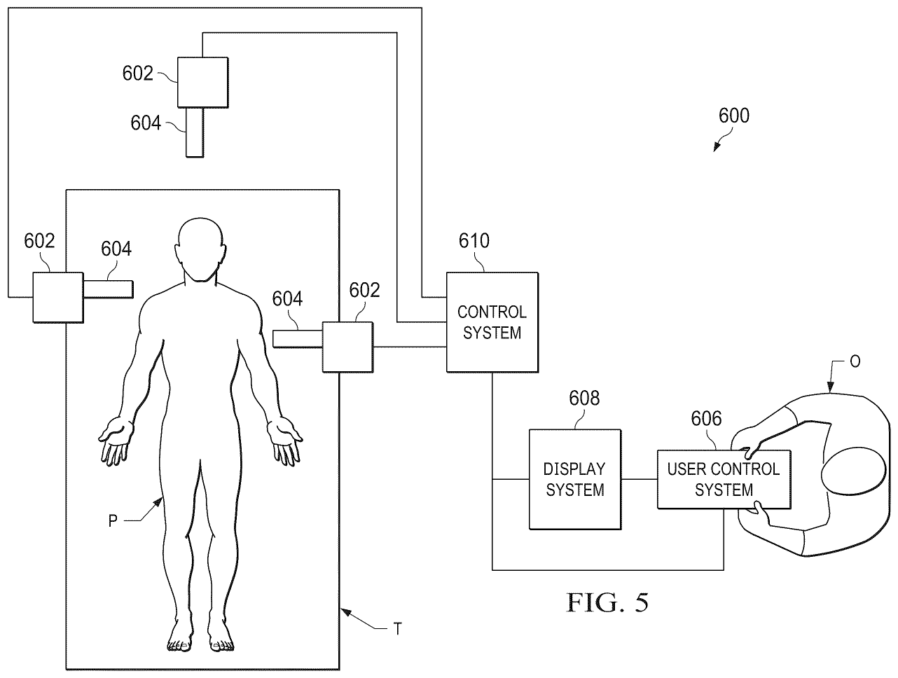

In some examples, the indicators 115 on the instrument holder frame 112 and the corresponding markings 155 on the cannula 150 may correspond to different medical procedures to be performed, or different states during a procedure. For example, if the medical procedure is to be performed on a bariatric patient or for a state of a procedure that indicates manipulation closer to the body wall, the control system (e.g., control system 610 in FIG. 5) may provide user instructions to insert the cannula 150 to a proximal-most marking 155A. The control system may then provide user instructions to align the proximal-most indicator 115A on the instrument holder frame 112 with the base marking 132 on the manipulator arm 130. In some examples, any of the other markings 155B-D may be used if the medical procedure is to be performed on a bariatric patient. The control system may provide user instructions regarding which marking to use for various medical procedures or procedure states based on the type of procedure to be performed, the state of the procedure, the body type of the patient, the type of instruments to be used, preselected user settings or preferences, the available operating space in the operating room, or any other related factors.

As discussed above, an adjustable connecting mechanism 145 may be used to adjust the position of the instrument holder 110 with respect to the manipulator arm 130. The adjustable connecting mechanism 145 may include a plurality of depth position settings 145A-145D. Various examples of adjustable connecting mechanisms will be described below. Other mechanically adjustable connecting mechanisms may be used alternatively or in addition to the adjustable connecting mechanisms described below. In some examples, any one or more of the adjustable connecting mechanisms described below may be motorized and may be controlled by the user and/or by a processing system/control system of a teleoperated system (e.g., teleoperated system 600 in FIG. 5), which includes the manipulator 130. The motor may adjust the position of the instrument holder frame 112 relative to the manipulator 130 based on the cannula insertion depth D₂. In some examples, any one or more of the adjustable connecting mechanisms described below may be manually adjusted by the user to adjust the position of the instrument holder frame 112 relative to the manipulator 130 based on the cannula insertion depth D₂.

FIG. 3A illustrates an adjustable connecting mechanism 200 (which may be the adjustable connecting mechanism 145) positioned at the connection location 140 of the manipulator arm 130. The adjustable connecting mechanism 200 includes a connection member 210, a channel 220 (e.g., a nut or a nut mount), and a lead screw 230. The lead screw 230 includes a proximal end cap 232, a distal end cap 234, and a plurality of depth position settings 236 (which may be the depth position settings 145A-D). The lead screw 230 is a threaded member and includes threads and grooves. As shown in FIG. 3A, the connection member 210 may be coupled to the instrument holder frame 112. The channel 220 may extend through the connection member 210, as shown in FIG. 3A. In some examples, the channel 220 may be coupled to the connection member 210 at any other location, such as to an exterior surface of the connection member 210 and/or to an end 212 of the connection member 210. The channel 220 is hollow and is sized to receive the lead screw 230. In some examples, the interior surface (not shown) of the channel 220 includes grooves sized and shaped to mate with and receive threads of the lead screw 230. In other examples, the interior surface of the channel 220 includes threads sized and shaped to mate with and be received by grooves of the lead screw 230.

The lead screw 230 may be rotated about its longitudinal axis A in a clockwise direction and a counterclockwise direction. Either one or both of the proximal end cap 232 and the distal end cap 234 may be used to rotate the lead screw 230. In some examples, the lead screw 230 may be coupled to a motor (not shown). The motor may be used to rotate the lead screw 230. Rotation of the lead screw 230 causes the instrument holder frame 112 to move via linear motion in the distal direction 101 or in the proximal direction 102. In some examples, when the lead screw 230 is rotated in a clockwise direction, the instrument holder frame 112 moves in the distal direction 101. In such examples, when the lead screw 230 is rotated in a counterclockwise direction, the instrument holder frame 112 moves in the proximal direction 102. In some examples, when the lead screw 230 is rotated in the counterclockwise direction, the instrument holder frame 112 moves in the proximal direction 102. In such examples, when the lead screw 230 is rotated in the clockwise direction, the instrument holder frame 112 moves in the distal direction 101. As discussed above, the instrument holder frame 112 moves relative to the manipulator arm 130. Therefore, when the lead screw 230 is rotated and the instrument holder frame 112 moves in the distal direction 101 or the proximal direction 102, the position of the manipulator arm 130 does not change.

As discussed above, the lead screw includes a plurality of depth position settings 236. The depth position settings 236 may be any type of indicator or marker displayed on the lead screw 230. The plurality of depth position settings 236 includes four individual depth position settings 236A, 236B, 236C, and 236D. Any other number of depth position settings may be included in the plurality of depth position settings 236. As shown in FIG. 3A, the depth position setting 236A is the most proximal depth position setting of the plurality of depth position setting 236, and the depth position setting 236D is the most distal depth position setting of the plurality of depth position setting 236. The depth position setting 236B is distal to the depth position setting 236A and proximal to the depth position setting 236C. The depth position setting 236C is proximal to the depth position setting 236D.

In some examples, the plurality of depth position settings 236 may be included on any one or more components of the adjustable connecting mechanism 200. For example, the depth position settings 236 may be included on the connection member 210. In some examples, the depth position settings 236 may be included on one or both of the manipulator arm 130 or the instrument holder frame 112.

Each of the depth position settings 236 corresponds to one of the indicators 115 on the instrument holder frame 112. For example, the most proximal depth position setting 236A of the depth position settings 236 corresponds to the most proximal indicator 115A of the indicators 115. The most distal depth position setting 236D corresponds to the most distal indicator 115D. Similarly, the depth position setting 236B corresponds to the indicator 115B, and the depth position setting 236C corresponds to the indicator 115C. In some examples, the depth position settings 236 may be labeled with different colors. Corresponding depth position settings and indicators may be the same color. For example, the depth position setting 236A and the indicator 115A may each be red; the depth position setting 236B and the indicator 115B may each be blue; the depth position setting 236C and the indicator 115C may each be green; and the depth position setting 236D and the indicator 115D may be each be yellow. These colors are exemplary only, and any other colors may be used for any of the corresponding pairs of depth position settings and indicators. Additionally or alternatively, the depth position settings 236 and the indicators 115 may be labeled with different graphical labels, such as different numbers (e.g., 1, 2, 3, and 4), patterns (e.g., dotted lines, dashed lines, etc.), shapes, words, or any combination thereof. Any other type of graphical labels may be used to identify the depth position settings 236 and the indicators 115.

Additionally or alternatively, each of the depth position settings 236 may correspond to one of the markings 155 on the cannula 150. The discussion above regarding how the different depth position settings 236 may correspond to the different indicators 115 similarly applies to how the different depth position settings 236 may correspond to the different markings 155.

In some examples, the depth position settings 236 may be detents on the lead screw 230. When the lead screw 230 is being rotated to adjust the position of the instrument holder 110 with respect to the manipulator arm 130, haptic feedback may be provided when a particular depth position setting 236A-D is aligned with the connection location 140 of the manipulator arm 130, for example. This may allow the user to more easily determine when a particular depth position setting is aligned with the connection location 140. This may then allow the user to more easily determine which indicator 115 on the instrument holder frame 112 is aligned with the base marking 132 on the manipulator arm 130.

FIG. 3B illustrates an adjustable connecting mechanism 300 (which may be the adjustable connecting mechanism 145) positioned at the connection location 140 of the manipulator arm 130. The adjustable connecting mechanism 300 is a rack and pinion assembly. As shown in FIG. 3B, the adjustable connecting mechanism 300 includes a rack 310, a pinion gear 320. The rack 310 includes multiple teeth 312, which may be coupled to the instrument holder frame 112, and multiple grooves 313 between the teeth 312. In some examples, the teeth 312 are coupled directly to an outer surface of the instrument holder frame 112. Alternatively, the teeth 312 may be coupled to a connection member (not shown), which is coupled to the instrument holder frame 112. The rack 310 also includes a plurality of depth position settings 314 (which may be the depth position settings 145A-D). The pinion gear 320 includes a hub 322 and teeth 324 extending radially outward from the hub 322. The teeth 324 are sized and shaped to mate with and extend into the grooves 313 between the teeth 312 of the rack 310.

The pinion gear 320 may be rotated about its center 326 in a clockwise direction and a counterclockwise direction. In some examples, the pinion gear 320 may be coupled to a motor (not shown). The motor may be used to rotate the pinion gear 320. Rotation of the pinion gear 320 causes the instrument holder frame 112 to move in the distal direction 101 or in the proximal direction 102. In some examples, when the pinion gear 320 is rotated in a clockwise direction, the instrument holder frame 112 moves in the distal direction 101. In some examples, when the pinion gear 320 is rotated in a counterclockwise direction, the instrument holder frame 112 moves in the proximal direction 102. As discussed above, the instrument holder frame 112 moves relative to the manipulator arm 130. Therefore, when the pinion gear 320 is rotated and the instrument holder frame 112 moves in the distal direction 101 or the proximal direction 102, the position of the manipulator arm 130 does not change.

As discussed above, the rack 310 includes a plurality of depth position settings 314. The depth position settings 314 may be any type of indicator or marker displayed on the rack 310. In some examples, the depth position settings 314 may be positioned within the grooves 313 between each of the teeth 312 of the rack 310, as shown in FIG. 3B. The plurality of depth position settings 314 includes four individual depth position settings 314A, 314B, 314C, and 314D. Any other number of depth position settings may be included in the plurality of depth position settings 314. As shown in FIG. 3B, the depth position setting 314A is the most proximal depth position setting of the plurality of depth position settings 314, and the depth position setting 314D is the most distal depth position setting of the plurality of depth position settings 314. The depth position setting 314B is distal to the depth position setting 314A and proximal to the depth position setting 314C. The depth position setting 314C is proximal to the depth position setting 314D.

In some examples, the plurality of depth position settings 314 may be included on any one or more components of the adjustable connecting mechanism 300. For example, the depth position settings 314 may be included on the pinion gear 320. In some examples, the depth position settings 314 may be included on one or both of the manipulator arm 130 or the instrument holder frame 112.

Each of the depth position settings 314 corresponds to one of the indicators 115 on the instrument holder frame 112. The details regarding how the depth position settings 314 correspond to the indicators 115 is similar to the discussion above with respect to FIG. 3A regarding how the depth position settings 236 correspond to the indicators 115. That discussion similarly applies to the depth position settings 314 and may be referenced when determining how the depth position settings 314 correspond to the indicators 115.

Additionally or alternatively, each of the depth position settings 314 may correspond to one of the markings 155 on the cannula 150. The discussion above with respect to FIG. 3A regarding how the different depth position settings 236 may correspond to the different indicators 115 similarly applies to how the different depth position settings 314 may correspond to the different markings 155.

FIG. 3C illustrates an adjustable connecting mechanism 400 (which may be the adjustable connecting mechanism 145) positioned at the connection location 140 of the manipulator arm 130. As shown in FIG. 3C, the adjustable connecting mechanism 400 includes teeth 410, grooves 411, and a ratchet assembly 420. The teeth 410 include a plurality of depth position settings 412 (which may be the depth position settings 145A-D). The teeth 410 may be coupled to the instrument holder frame 112. In some examples, the teeth 410 are coupled directly to an outer surface of the instrument holder frame 112. Alternatively, the teeth 410 may be coupled to a connection member (not shown), which is coupled to the instrument holder frame 112. The ratchet assembly 420 includes a tab 422 and a key 424.

The tab 422 may be depressed in a direction that is radially inward toward an axis A of the instrument holder frame 112. When the tab 422 is depressed, the key 424 may be moved radially outward. In some examples, the tab 422 may include an internal biasing member, such as a spring mechanism (not shown), that biases the tab 422 in a radially outward direction. For example, when the tab 422 is not depressed, the tab 422 may remain in an extended position due to a biasing force imparted by the internal biasing member.

In some examples, the ratchet assembly 420 may be movable between an engaged configuration and a disengaged configuration. In the engaged configuration, the tab 422 is not depressed, and the key 424 is engaged with at least one tooth 410 on the instrument holder frame 112. In some examples, when the ratchet assembly 420 is in the engaged configuration, the instrument holder frame 112 is not movable in the distal direction 101. In such examples, when the ratchet assembly 420 is in the engaged configuration, the instrument holder frame 112 is in a locked configuration. When the ratchet assembly 420 is in the disengaged configuration, the tab 422 is depressed, and the key 424 is disengaged from the teeth 410. When the ratchet assembly 420 is in the disengaged configuration, the instrument holder frame 112 is movable in the distal direction 101. Thus, when the ratchet assembly 420 is in the disengaged configuration, the instrument holder frame 112 is in an unlocked configuration. In some examples, when the instrument holder frame 112 is pushed in the proximal direction 102, the key 424 slides over the teeth 410 even if the tab 422 is not depressed. Therefore, to move the instrument holder frame 112 in the distal direction 101, the tab 422 is depressed, and the instrument holder frame 112 is pulled in the distal direction 101. To move the instrument holder frame 112 in the proximal direction 102, though, the tab 422 does not need to be depressed (but may still be depressed in some examples), and the instrument holder frame 112 is pushed in the proximal direction 102. In some examples, the ratchet assembly 420 may be coupled to a motor (not shown). The motor may be used to move the instrument holder frame 112. For example, when the ratchet assembly 420 is in the disengaged configuration, the motor may move the instrument holder frame 112 in the distal direction 101. The motor may also push the instrument holder frame 112 in the proximal direction 102.

In some alternative examples, when the ratchet assembly 420 is in the engaged configuration, the instrument holder frame 112 is not movable in the proximal direction 102. In such examples, when the ratchet assembly 420 is in the engaged configuration, the instrument holder frame 112 is in a locked configuration. When the ratchet assembly 420 is in the disengaged configuration, the tab 422 is depressed, and the key 424 is disengaged from the teeth 410. When the ratchet assembly 420 is in the disengaged configuration, the instrument holder frame 112 is movable in the proximal direction 102. Thus, when the ratchet assembly 420 is in the disengaged configuration, the instrument holder frame 112 is in an unlocked configuration. In some examples, when the instrument holder frame 112 is pushed in the distal direction 101, the key 424 slides over the teeth 410 even if the tab 422 is not depressed. Therefore, to move the instrument holder frame 112 in the proximal direction 102, the tab 422 is depressed, and the instrument holder frame 112 is pulled in the proximal direction 102. To move the instrument holder frame 112 in the distal direction 101, though, the tab 422 does not need to be depressed (but may still be depressed in some examples), and the instrument holder frame 112 is pushed in the distal direction 101. In some examples, the ratchet assembly 420 may be coupled to a motor (not shown). The motor may be used to move the instrument holder frame 112. For example, when the ratchet assembly 420 is in the disengaged configuration, the motor may move the instrument holder frame 112 in the proximal direction 102. The motor may also push the instrument holder frame 112 in the distal direction 101.

As discussed above, the teeth 410 include a plurality of depth position settings 412. The depth position settings 412 may be any type of indicator or marker displayed on the teeth 410. In some examples, the depth position settings 412 may be positioned within the grooves 411 between each of the teeth 410, as shown in FIG. 3C. The plurality of depth position settings 412 includes four individual depth position settings 412A, 412B, 412C, and 412D. Any other number of depth position settings may be included in the plurality of depth position settings 412. As shown in FIG. 3C, the depth position setting 412A is the most proximal depth position setting of the plurality of depth position settings 412, and the depth position setting 412D is the most distal depth position setting of the plurality of depth position settings 412. The depth position setting 412B is distal to the depth position setting 412A and proximal to the depth position setting 412C. The depth position setting 412C is proximal to the depth position setting 412D.

In some examples, the plurality of depth position settings 412 may be included on any one or more components of the adjustable connecting mechanism 400. For example, the depth position settings 412 may be included on the teeth 410 and/or on the ratchet assembly 420. In some examples, the depth position settings 412 may be included on one or both of the manipulator arm 130 or the instrument holder frame 112.

Each of the depth position settings 412 corresponds to one of the indicators 115 on the instrument holder frame 112. The details regarding how the depth position settings 412 correspond to the indicators 115 is similar to the discussion above with respect to FIG. 3A regarding how the depth position settings 236 correspond to the indicators 115. That discussion similarly applies to the depth position settings 412 and may be referenced when determining how the depth position settings 412 correspond to the indicators 115.

Additionally or alternatively, each of the depth position settings 412 may correspond to one of the markings 155 on the cannula 150. The discussion above with respect to FIG. 3A regarding how the different depth position settings 236 may correspond to the different indicators 115 similarly applies to how the different depth position settings 412 may correspond to the different markings 155.

As discussed above, the instrument holder frame 112 moves relative to the manipulator arm 130. Therefore, when the instrument holder frame 112 moves in the distal direction 101 or the proximal direction 102, the position of the manipulator arm 130 does not change.

In some examples, any one or more of the adjustable connecting mechanism (e.g., the adjustable connecting mechanism 200, 300, 400), the manipulator arm 130, or the instrument holder frame 112 may include one or more position sensors that are used to measure the position of the instrument holder frame 112 with respect to the manipulator arm 130. For example, the control system of the teleoperated system may receive information from the position sensors to determine the relative position between the instrument holder frame 112 and the manipulator arm 130. In some examples, a separate position sensor may be positioned at each indicator 115 on the instrument holder frame 112. Additionally or alternatively, a separate RFID chip may be placed at each indicator 115 on the instrument holder frame 112. The base marking 132 on the manipulator arm 130 may include an RFID reader device that may identify the corresponding RFID chip in the instrument holder frame 112 when each successive indicator 115A-115D on the instrument holder frame 112 is aligned with and/or passes the base marking 132 on the manipulator arm 130.

In some examples, the manipulation system 100 may include one or more force sensors that may determine the force exerted on the manipulator arm 130 and/or the instrument holder 110 (e.g., gravitational forces). The force sensors may be included on any one or more of the adjustable connecting mechanism (e.g., the adjustable connecting mechanism 200, 300, 400), the manipulator arm 130, or the instrument holder frame 112. The force data may be received by a control system of the manipulation system 100. Based on the force data, the control system may estimate a position at which the anatomical interaction location of the cannula 150 should be set. The control system may provide user instructions to insert the cannula 150 to a specific cannula insertion depth D₂ based on the estimated position of the anatomical interaction location of the cannula 150.

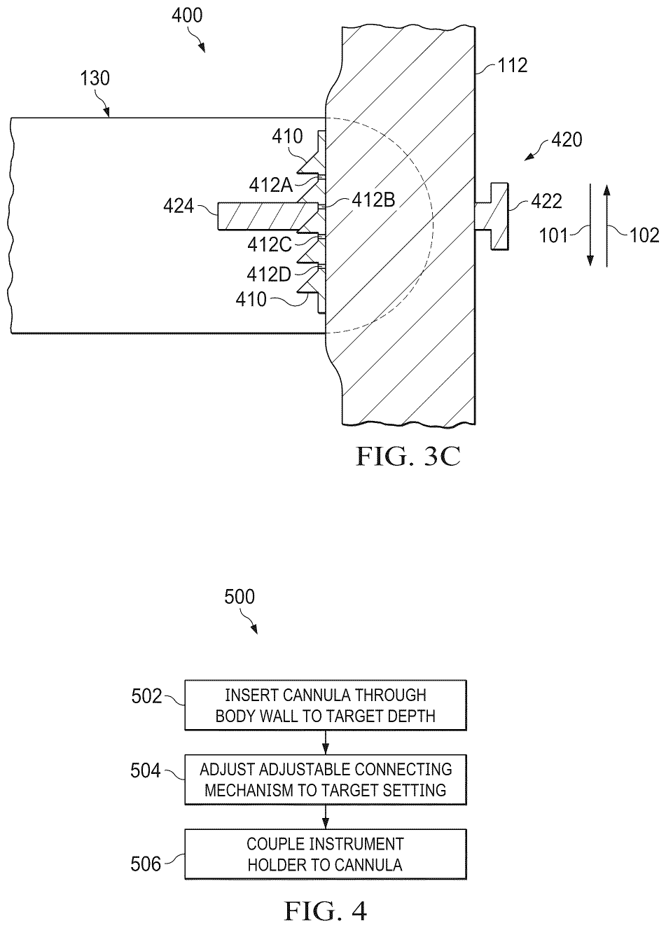

FIG. 4 is a flowchart illustrating a method 500 of positioning a manipulator 130 and a cannula 150 relative to a body wall 160 of a patient. The method 500 is illustrated as a set of operations or processes 502 through 506. The processes illustrated in FIG. 4 may be performed in a different order than the order shown in FIG. 4, and one or more of the illustrated processes might not be performed in some examples of the method 500. Additionally, one or more processes that are not expressly illustrated in FIG. 4 may be included before, after, in between, or as part of the illustrated processes.

At a process 502, a cannula (e.g., the cannula 150) is inserted through a body wall (e.g., the body wall 160) of a patient to a target depth. The target depth may be determined based on the type of procedure to be performed, the state of the procedure, the type of instruments to be used, the body type of the patient, preselected user settings or preferences, the available operating space in the operating room, or any other related factors. When the cannula 150 is positioned at the target depth, one of the markings 155 on the cannula 150 is positioned at the remote center 170. The marking positioned at the remote center 170 may indicate the anatomical interaction location of the cannula 150.

At a process 504, an adjustable connecting mechanism (e.g., any one of the adjustable connecting mechanisms 200, 300, 400) is adjusted to a target setting. When the adjustable connecting mechanism is adjusted, the instrument holder 110 is adjusted relative to the manipulator arm 130. For example, the instrument holder 110 may be moved in a distal direction towards the body wall 160 of the patient. When the adjustable connecting mechanism is positioned at the target setting, one of the indicators 115 on the instrument holder frame 112 is aligned with the base marking 132 on the manipulator arm 130. The indicator that is aligned with the base marking 132 may be the indicator that corresponds to the marking on the cannula 150 positioned at the remote center 170, as discussed above.

At a process 506, the instrument holder 110 is coupled to the cannula 150. For example, the connection portion 114 of the instrument holder 110 may be coupled to the proximal portion 152 of the cannula 150. Before, during, or after coupling the instrument holder 110 with the cannula 150, the instrument shaft 126 may be inserted through the cannula 150 and into the patient (e.g., into the interior space 164).

In some examples, the components discussed above may be used in a procedure performed with a teleoperated system as described in further detail below. The teleoperated system may be suitable for use in, for example, medical, teleoperated medical, surgical, diagnostic, therapeutic, or biopsy procedures. While some examples are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic, general teleoperational, or robotic medical systems.

As shown in FIG. 5, a teleoperated system 600 generally includes at least one manipulator assembly 602 (e.g., the manipulator assembly 130). In some examples, the manipulator assembly 602 or components of the manipulator assembly 602 may be the manipulator 130. Although three manipulator assemblies 602 are illustrated in the example of FIG. 5, in other examples, more or fewer manipulator assemblies may be used. The exact number of manipulator assemblies will depend on the medical procedure and the space constraints within the operating room, among other factors.

The manipulator assembly 602 is used to operate a medical instrument 604 (e.g., a surgical instrument or an image capturing device) in performing various procedures on a patient P. The manipulator assembly 602 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. In some examples, the manipulator assembly 602 may be mounted to or near an operating or surgical table T. In such examples, the manipulator assembly 602 may be mounted directly to the table T or to a rail coupled to the table T. In various other examples, the manipulator assembly 602 may be mounted to a manipulating system (e.g., a movable floor supported patient-side cart). The manipulating system may be separate from and spaced from the table T in the operating room. In such examples, the manipulating system may be independently movable relative to the table T. In other examples, the manipulator assembly 602 may be mounted to a ceiling of the operating room. In some additional examples, the manipulator assembly 602 may be mounted to one or more of a floor of the operating room or a wall of the operating room. In examples in which a plurality of manipulator assemblies 602 are employed, one or more of the manipulator assemblies 602 may support medical instruments, and another of the manipulator assemblies may support an image capturing device such as a monoscopic or stereoscopic endoscope. In such examples, one or more of the manipulator assemblies 602 may be mounted to any structure or in any manner as described above. For example, one manipulator assembly 602 may be mounted to the table T and another manipulator assembly 602 may be mounted to a manipulating system. In other examples, an additional manipulator assembly 602 may be mounted to the ceiling of the operating room.

A user control system 606 allows an operator O (e.g., a surgeon or other clinician, as illustrated in FIG. 6) to view the interventional site and to control manipulator assembly 602. In some examples, the user control system 606 is a surgeon console, which is usually located in the same room as the operating or surgical table T, such as at the side of a table on which patient P is located. However, it is to be understood that operator O can be located in a different room or a completely different building from patient P. Additionally, multiple user control systems may be collocated or they may be positioned in separate locations. Multiple user control systems allow more than one operator to control one or more teleoperated manipulator assemblies in various combinations. User control system 606 generally includes one or more input devices for controlling manipulator assembly 602. The input devices may include any number of a variety of devices, such as joysticks, trackballs, data gloves, trigger-guns, hand-operated controllers, voice recognition devices, body motion or presence sensors, and/or the like. To provide operator O a strong sense of directly controlling medical instrument 604, the input devices may be provided with the same degrees of freedom as the associated medical instrument 604. In this manner, the input devices provide operator O with telepresence or the perception that the input devices are integral with medical instrument 604.

In some examples, the input devices may have more or fewer degrees of freedom than the associated medical instrument 604 and still provide operator O with telepresence. In some examples, the input devices may optionally be manual input devices which move with six degrees of freedom, and which may also include an actuatable handle for actuating instruments (for example, for closing grasping jaws, applying an electrical potential to an electrode, delivering a medicinal treatment, and/or the like).

Manipulator assembly 602 supports medical instrument 604 and may include a kinematic structure of one or more non-servo controlled links (e.g., one or more links that may be manually positioned and locked in place, generally referred to as a set-up structure), and/or one or more servo controlled links (e.g., one or more links that may be controlled in response to commands from a control system), and a manipulator. Manipulator assembly 602 may optionally include a plurality of actuators or motors that drive inputs on medical instrument 604 in response to commands from the control system (e.g., a control system 610). The actuators may optionally include drive systems that when coupled to medical instrument 604 may advance medical instrument 604 into a naturally or surgically created anatomic orifice. Other drive systems May move the distal end of medical instrument 604 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulable end effector of medical instrument 604 for grasping tissue in the jaws of a biopsy device and/or the like. Actuator position sensors such as resolvers, encoders, potentiometers, and other mechanisms may provide sensor data to system 600 describing the rotation and orientation of the motor shafts. This position sensor data may be used to determine motion of the objects manipulated by the actuators. The manipulator assembly 602 may position its held instrument 604 so that a pivot point occurs at the instrument's entry aperture into the patient. The manipulator assembly 602 may then manipulate its held instrument so that the instrument may be pivoted about the pivot point, inserted into and retracted out of the entry aperture, and rotated about its shaft axis.

System 600 also includes a display system 608 for displaying an image or representation of the surgical site and medical instrument 604. Display system 608 and user control system 606 may be oriented so operator O can control medical instrument 604 and user control system 606 with the perception of telepresence. In some examples, medical instrument 604 may include a visualization system, which may include a viewing scope assembly that records a concurrent or real-time image of a surgical site and provides the image to the operator O and/or other operators or personnel through one or more displays of system 600, such as one or more displays of display system 608. The concurrent image may be, for example, a two or three dimensional image captured by an endoscope positioned within the surgical site. The visualization system may be implemented as hardware, firmware, software, or a combination thereof which interact with or are otherwise executed by one or more computer processors, which may include the processors of a control system 610.

In some examples, display system 608 may present images of a surgical site recorded pre-operatively or intra-operatively using image data from imaging technology such as, computed tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The pre-operative or intra-operative image data may be presented as two-dimensional, three-dimensional, or four-dimensional (including, e.g., time-based or velocity-based information) images and/or as images from models created from the pre-operative or intra-operative image data sets.

System 600 may also include control system 610. Control system 610 includes at least one memory and at least one computer processor (not shown) for effecting control between medical instrument 604, user control system 606, and display system 608. Control system 610 also includes programmed instructions (e.g., a non-transitory machine-readable medium storing the instructions) to implement some or all the methods described in accordance with aspects disclosed herein, including instructions for providing information to display system 608. While control system 610 is shown as a single block in the simplified schematic of FIG. 6, the system may include two or more data processing circuits with one portion of the processing optionally being performed on or adjacent to manipulator assembly 602, another portion of the processing being performed at user control system 606, and/or the like. The processors of control system 610 may execute instructions, which may be computer readable instructions, comprising instruction corresponding to processes disclosed herein and described in more detail below. Any of a wide variety of centralized or distributed data processing architectures may be employed. Similarly, the programmed instructions may be implemented as a number of separate programs or subroutines, or they may be integrated into a number of other aspects of the robotic medical systems described herein. In one example, control system 610 supports wireless communication protocols such as Bluetooth, IrDA, HomeRF, IEEE 802.11, DECT, and Wireless Telemetry.

Movement of a manipulator assembly 602 may be controlled by the control system 610 so that a shaft or intermediate portion of instruments mounted to the manipulator assemblies 602 are constrained to safe motions through minimally invasive surgical access sites or other apertures. Such motion may include, for example, axial insertion of a shaft through an aperture site, rotation of the shaft about its axis, and pivotal motion of the shaft about a pivot point adjacent the access site. In some cases, excessive lateral motion of the shaft that might otherwise tear the tissues adjacent the aperture or enlarge the access site inadvertently is inhibited. Some or all of such constraint on the motions of the manipulator assemblies 602 at the access sites may be imposed using mechanical manipulator joint linkages that inhibit improper motions, or may in part or in full be imposed using data processing and control techniques. In some examples, control system 610 may receive force and/or torque feedback from medical instrument 604. Responsive to the feedback, control system 610 may transmit signals to user control system 606. In some examples, control system 610 may transmit signals instructing one or more actuators of manipulator assembly 602 to move medical instrument 604.

In some examples, the manipulator assemblies 602 may be used in a medical procedure. In other examples, the manipulator assemblies 602 may be used in procedures involving traditional manually operated minimally invasive surgical instruments, such as manual laparoscopy. While only three manipulator assemblies 602 are depicted, it is to be understood that more than three (e.g., four, five, six, and more than six) or fewer than three (e.g., one or two) manipulator assemblies can be included in some configurations.

In some examples, an equipment rail is attached to the table T. One or more of the manipulator assemblies 602 may be coupled to the equipment rail during the medical procedure. The manipulator assemblies 602 may be coupled to the equipment rail after being fully assembled, or the manipulator assemblies 602 may be coupled to the equipment rail before being fully assembled.

In addition, the singular forms “a”, “an”, and “the” are intended to include the plural forms as well, unless the context indicates otherwise. And the terms “comprises,” “comprising,” “includes,” “has,” and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. The auxiliary verb “may” likewise implies that a feature, step, operation, element, or component is optional.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system”, are analogous.

Although some of the examples described herein refer to surgical procedures or instruments, or medical procedures and medical instruments, the techniques disclosed apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy), and performing procedures on human or animal cadavers. Further, these techniques can also be used for surgical and nonsurgical medical treatment or diagnosis procedures.

Further, although some of the examples presented in this disclosure discuss teleoperational robotic systems or remotely operable systems, the techniques disclosed are also applicable to computer-assisted systems that are directly and manually moved by operators, in part or in whole.

Additionally, one or more elements in examples of this disclosure may be implemented in software to execute on a processor of a computer system such as a control processing system. When implemented in software, the elements of the examples of the present disclosure are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc.

Note that the processes and displays presented may not inherently be related to any particular computer or other apparatus, and various systems may be used with programs in accordance with the teachings herein. The required structure for a variety of the systems discussed above will appear as elements in the claims. In addition, the examples of the present disclosure are not described with reference to any particular programming language. It will be appreciated that a variety of programming languages may be used to implement the teachings of the present disclosure as described herein.

While certain examples of the present disclosure have been described and shown in the accompanying drawings, it is to be understood that such examples are merely illustrative of and not restrictive to the broad disclosed concepts, and that the examples of the present disclosure not be limited to the specific constructions and arrangements shown and described, since various other modifications may occur to those ordinarily skilled in the art.

1. A method for operating a medical system, the method comprising:
inserting a cannula through a body wall of a patient to a target depth;
adjusting an adjustable connecting mechanism to a target setting to position an instrument holder at a target position relative to a robotic manipulator arm, the adjustable connecting mechanism comprising a plurality of depth position settings to adjust a position of the instrument holder relative to the robotic manipulator arm based on the target depth, the target setting corresponding to the target depth, wherein a remote center distance between the robotic manipulator arm and a remote center of motion is maintained as the adjustable connecting mechanism moves the instrument holder to the target position relative to the robotic manipulator arm, and wherein the cannula pivots about the remote center of motion; and
coupling the instrument holder to the cannula.

2. (canceled)

3. The method of claim 1, wherein the adjustable connecting mechanism includes a lead screw.

4. The method of claim 1, wherein the adjustable connecting mechanism includes a rack and pinion.

5. The method of claim 1, wherein the adjustable connecting mechanism includes a ratchet.

6. The method of claim 1, wherein the adjustable connecting mechanism includes a motor, and wherein the motor adjusts the position of the instrument holder relative to the robotic manipulator arm.

7. The method of claim 1, wherein the cannula includes a plurality of markings, and wherein each marking of the plurality of markings indicates a different insertion depth at which the cannula may be inserted into the body wall.

8. The method of claim 7, wherein at least one of the plurality of markings corresponds to the target depth.

9. The method of claim 7, wherein each marking of the plurality of markings identifies a target insertion depth for one or more different medical procedures or for one or more different states during a procedure.

10. The method of claim 1, wherein the instrument holder includes a plurality of indicators on an outer surface of the instrument holder, and wherein each indicator of the plurality of indicators designates a different position of the instrument holder relative to the robotic manipulator arm.

11. The method of claim 10, wherein at least one indicator of the plurality of indicators corresponds to the target setting.

12. The method of claim 1, further comprising detecting, by a position sensor of the robotic manipulator arm, a position of the instrument holder relative to the robotic manipulator arm.

13. The method of claim 1, further comprising detecting, by a position sensor of the instrument holder, a position of the instrument holder relative to the robotic manipulator arm.

14. The method of claim 1, further comprising:
receiving force data from one or more force sensors of the robotic manipulator arm, the force data indicating one or more forces acting on the robotic manipulator arm; and
determining the target depth of the cannula based on the force data.

15. A system comprising:
a robotic manipulator arm;
an instrument holder coupled to the robotic manipulator arm by an adjustable connecting mechanism;
a processing system including one or more processors; and
a memory having computer readable instructions stored thereon which, when executed by the one or more processors, cause the processing system to:
provide user instructions for inserting a cannula through a body wall of a patient to a target depth;
provide user instructions for adjusting the adjustable connecting mechanism to a first setting to position the instrument holder at a first position relative to the robotic manipulator arm, the adjustable connecting mechanism including a plurality of depth position settings to adjust a position of the instrument holder relative to the robotic manipulator arm based on the target depth, the first setting corresponding to the target depth, wherein a remote center distance between the robotic manipulator arm and a remote center of motion is maintained as the adjustable connecting mechanism moves the instrument holder to the first position relative to the robotic manipulator arm, and wherein the cannula pivots about the remote center of motion; and
provide user instructions for coupling the instrument holder to the cannula.

16-21. (canceled)

22. The system of claim 15, wherein the cannula includes a plurality of markings, wherein each marking of the plurality of markings indicates a different insertion depth at which the cannula may be inserted into the body wall, and wherein the instrument holder includes a plurality of indicators on an outer surface of the instrument holder, wherein each indicator of the plurality of indicators designates a different position of the instrument holder relative to the robotic manipulator arm.

23-29. (canceled)

30. A system comprising:
a robotic manipulator arm comprising:
a hardware constrained remote center of motion;
an instrument holder for coupling to an instrument; and
a connecting mechanism coupling the robotic manipulator arm to the instrument holder; and
a cannula,
wherein the connecting mechanism is configured to adjust a position of the instrument holder relative to the robotic manipulator arm between a first position and a second position, the first position corresponding to a first insertion depth of the cannula in a patient body wall, the second position corresponding to a second insertion depth of the cannula in the patient body wall, wherein the remote center of motion relative to the patient body wall is maintained when the instrument holder is in the first position and in the second position.

31-34. (canceled)

35. The system of claim 30, wherein the cannula includes a plurality of markings, wherein each marking of the plurality of markings indicates a different insertion depth at which the cannula may be inserted into the patient body wall, and wherein at least one of the plurality of markings corresponds to the first insertion depth.

36. (canceled)

37. (canceled)

38. The system of claim 30, wherein the instrument holder includes a plurality of indicators on an outer surface of the instrument holder, wherein each indicator of the plurality of indicators designates a different position of the instrument holder relative to the robotic manipulator arm, and wherein at least one indicator of the plurality of indicators corresponds to the first insertion depth.

39. (canceled)

40. The system of claim 30, wherein the robotic manipulator arm includes a position sensor configured to detect a position of the instrument holder relative to the robotic manipulator arm.

41. The system of claim 30, wherein the instrument holder includes a position sensor configured to detect a position of the instrument holder relative to the robotic manipulator arm.
